(19) United States
(12) Patent Application Publication
(10) Pub. No.: US 2009/0074838 A1
(43) Pub. Date: Mar. 19, 2009

(54) MEDICAL DEVICES HAVING BIOERODABLE LAYERS FOR THE RELEASE OF THERAPEUTIC AGENTS

(75) Inventors: Courtney Sikes, Uxbridge, MA (US); Mark Steckel, Sharon, MA (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/056,689

(22) Filed: Mar. 27, 2008

Related U.S. Application Data
(60) Provisional application No. 60/920,394, filed on Mar. 28, 2007.

(51) Int. Cl. A61F 2/04

(52) U.S. Cl. 424/426

(57) ABSTRACT

According to an aspect of the present invention, medical devices are provided which comprise: (a) a substrate, (b) an inner bioerodable polymeric layer over the substrate that comprises (i) 80 wt % or more of an amorphous biodegradable polymeric component and (ii) 20 wt % or less of a therapeutic agent component, and (c) an outer bioerodable polymeric layer over the inner bioerodable polymeric layer that comprises (i) 80 wt % or more of an amorphous biodegradable polymeric component and (ii) 20 wt % or less of a therapeutic agent component. The compositions of the inner and outer layers differ such that the outer layer has a bioerosion rate that is faster than that of the inner layer.

### RELATED APPLICATION SECTION

This application claims the benefit of U.S. Provisional Patent Application Ser. No. 60/920,394, filed Mar. 28, 2007, entitled “Medical Devices Having Biodegradable Layers for the Release of Therapeutic Agents”, which is incorporated by reference herein in its entirety.

### FIELD OF THE INVENTION

The present invention relates to medical devices which are a least partially biodegradable and which release therapeutic agents.

### BACKGROUND

Numerous polymer-based medical devices have been developed for implantation or insertion into the body. For example, in recent years, drug eluting coronary stents, which are commercially available from Boston Scientific Corp. (TAXUS), Johnson & Johnson (CYPHER) and others, have become the standard of care for maintaining vessel patency. These existing products are based on metallic balloon expandable stents with biostable polymer coatings, which release antiproliferative drugs at a controlled rate and total dose.

Specific examples of biostable polymers for drug eluting polymer coatings include block copolymers of polyisobutylene and polystyrene, for example, poly(styrene-b-isobutylene-b-styrene triblock copolymers (SIBS copolymers), which are described in U.S. Pat. No. 6,545,097 to Pinchuk et al. and have proven valuable in implantable and insertable medical devices for a variety of reasons, including their excellent elasticity, strength and biocompatibility. SIBS copolymer systems are also effective drug delivery systems for providing therapeutic agents to sites in vivo, as evidenced by the TAXUS products currently being sold by Boston Scientific, for example, TAXUS EXPRESS SR stents, which contain a coating of 8.8 wt % paclitaxel and 91.2 wt % SIBS on a stainless steel coronary stent. The elution profile of this stent is illustrated in curve a of FIG. 1. As can be seen, drug continues to be released from the stent in small quantities for a period of at least two months. Moreover, a majority of the drug remains trapped within the device after two months, and continues to elute over time. Although not immediately apparent due to the scale of FIG. 1, the TAXUS EXPRESS SR stents produce a burst of paclitaxel in the early stages. This can be better seen from FIG. 2, which illustrates the release profile of an analogous stent coating that contains 25 wt % paclitaxel and 75 wt % SIBS. FIG. 2 is taken from U.S. Ser. No. 11/048,613, filed Feb. 1, 2005.

Biodegradable polymers have certain benefits over biostable polymers such as SIBS. For example, the issue of long-term drug entrapment and release is addressed. Moreover, because they erode over time, biodegradable polymers have the potential to reduce or eliminate long term effects that may be associated with non-biodegradable polymers (e.g.,, foreign body effects, etc.).

### SUMMARY OF THE INVENTION

According to an aspect of the present invention, medical devices are provided which comprise: (a) a substrate, (b) an inner bioerodable polymeric layer over the substrate that comprises (i) 80 wt % or more of an amorphous biodegradable polymeric component and (ii) 20 wt % or less of a therapeutic agent component, and (c) an outer bioerodable polymeric layer over the inner bioerodable polymeric layer that comprises (i) 80 wt % or more of an amorphous biodegradable polymeric component and (ii) 20 wt % or less of a therapeutic agent component. The compositions of the inner and outer bioerodable polymeric layers differ such that the outer bioerodable polymeric layer has a bioerosion rate that is faster than that of the inner bioerodable polymeric layer.

Further aspects of the present invention are enumerated below:

Aspect 1. A medical device comprising (a) a substrate, (b) an inner biocrodable polymeric layer over the substrate that comprises (i) 80 wt % or more of a first amorphous biodegradable polymeric component and (ii) 20 wt % or less of a therapeutic agent component, and (c) an outer bioerodable polymeric layer over the inner bioerodable polymeric layer that comprises (i) 80 wt % or more of a second amorphous biodegradable polymeric component and (ii) 20 wt % or less of the therapeutic agent component, wherein the first and second amorphous biodegradable polymeric components are the same or different, and wherein the inner and outer bioerodable polymeric layers differ in composition such that the outer bioerodable polymeric layer has a bioerosion rate that is faster than that of the inner bioerodable polymeric layer.

Aspect 2. The medical device of Aspect 1, wherein the first and second amorphous biodegradable polymeric components are different.

Aspect 3. The medical device of Aspect 2, wherein the first amorphous biodegradable polymeric component comprises a first polymer having a first monomer content and wherein the second amorphous biodegradable polymeric component comprises a second polymer having a second monomer content that differs from the first monomer content.

Aspect 4. The medical device of Aspect 3, wherein the first polymer comprises a monomer species that is not found in the second polymer or wherein the second polymer comprises a monomer species that is not found in the first polymer.

Aspect 5. The medical device of Aspect 4, wherein the first polymer is poly(1-lactic acid-co-glycolic acid) or poly(d,l-lactic acid-co-glycolic acid) and wherein the second polymer is poly(d,l-lactic acid).

Aspect 6. The medical device of Aspect 3, wherein the first and second polymers are copolymers comprising first and second monomers and wherein the ratio of the first monomer to the second monomer differs between the first and second polymers.

Aspect 7. The medical device of Aspect 6, wherein the first and second polymers are poly(1-lactic acid-co-glycolic acid) or poly(d,l-lactic acid-co-glycolic acid), and wherein the mol % of lactic acid within the first polymer is less than that within the second polymer.

Aspect 8. The medical device of Aspect 7, wherein the amount of d,l-lactic acid within the first polymer ranges from 30 to 85 mol % and wherein the amount of d,l-lactic acid within the second polymer ranges from 30 to 100 mol %.

Aspect 9. The medical device of Aspect 1, wherein the first and second amorphous biodegradable polymeric components are the same and wherein the wt % of the therapeutic agent component within the outer bioerodable polymeric layer is greater than that of the inner bioerodable polymeric layer.

Aspect 10. The medical device of Aspect 1, wherein the first and second amorphous biodegradable polymeric components are different and wherein the wt % of the therapeutic agent within the outer bioerodable polymeric layer is greater than that of the inner bioerodable polymeric layer.

Aspect 11. The medical device of Aspect 1, wherein the inner and outer layers each comprises 10 wt % or less of the therapeutic agent component.

Aspect 12. The medical device of Aspect 1, wherein the wt % of the therapeutic agent component within the outer bioerodable polymeric layer is at least 2 times that of the inner bioerodable polymeric layer.

Aspect 13. The medical device of Aspect 1, wherein the outer bioerodable polymeric layer is the outermost layer in the medical device.

Aspect 14. The medical device of Aspect 1, wherein no region within the medical device comprises a crystalline or semi-crystalline biodegradable polymeric component.

Aspect 15. The medical device of Aspect 1, wherein the substrate is metallic.

Aspect 16. The medical device of Aspect 1, wherein the substrate is a vascular stent.

Aspect 17. The medical device of Aspect 1, wherein the device comprises no polymeric layers other than the inner and outer bioerodable polymeric layers.

Aspect 18. The medical device of Aspect 1, wherein the inner and outer bioerodable polymeric layers are non-porous.

Aspect 19. The medical device of Aspect 1, wherein the first and second amorphous biodegradable polymeric components consist of biodegradable polyesters.

Aspect 20. The medical device of Aspect 19, wherein the biodegradable polyesters comprise lactic acid monomers or a combination of lactic acid and glycolic acid monomers.

Aspect 21. The medical device of Aspect 1, wherein the therapeutic agent component comprises a plurality of differing therapeutic agents.

Aspect 22. The medical device of Aspect 1, wherein the therapeutic agent component is selected from anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, agents that interfere with endogenous vasoactive mechanisms, and combinations thereof.

Aspect 23. The medical device of Aspect 1, wherein the medical device is a vascular medical device.

Aspect 24. The medical device of Aspect 2, wherein the first amorphous biodegradable polymeric component comprises a first polymer having a first monomer content, wherein the second amorphous biodegradable polymeric component comprises a second polymer having a second monomer content that is the same as the first monomer content, and wherein the second polymer has a number average molecular weight that is at least 10 kDa greater than the first polymer.

Other aspects and embodiments of the invention, as well as various advantages of the same will become immediately apparent to those of ordinary skill in the art upon reading the disclosure to follow.

### BRIEF DESCRIPTION OF THE DRAWINGS

FIGS. 1 and 2 are plots of percent paclicaxel release as a function of time for various stent compositions.

FIG. 3 is a schematic view of a medical device or portion thereof that is substantially rectangular in cross-section, in accordance with an embodiment of the invention.

FIG. 4 is a schematic partial cross-sectional view of a substantially planar medical device or portion thereof, in accordance with an embodiment of the invention.

FIG. 5 is a schematic view of a medical device or portion thereof that is substantially annular in cross-section, in accordance with an embodiment of the invention.

FIG. 6 is a schematic partial cross-sectional view of a substantially planar medical device or portion thereof, in accordance with an embodiment of the invention.

FIG. 7 is a schematic perspective view of a coronary stent, in accordance with an embodiment of the invention.

FIG. 8 contains hypothetical plots of percent drug release as a function of time associated with a medical device having an inner bioerodable drug-containing layer and an outer bioerodable drug-containing layer, with drug release illustrated for the inner layer, the outer layer, and the combined release of the inner and outer layers.

### DETAILED DESCRIPTION OF THE INVENTION

According to an aspect of the present invention, medical devices are provided which comprise: (a) a substrate, (b) an inner bioerodable polymeric layer (also referred to herein as an “inner layer”) over the substrate that comprises (i) 80 wt % or more of an amorphous biodegradable polymeric component (also referred to herein as a “polymeric component”) and (ii) 20 wt % or less of a therapeutic agent component, and (c) an outer bioerodable polymeric layer (also referred to herein as an “outer layer”) over the inner bioerodable polymeric layer that comprises (i) 80 wt % or more of an amorphous biodegradable polymeric component and (ii) 20 wt % or less of a therapeutic agent component. The compositions of the inner and outer layers differ such that the outer layer has a biocrosion rate that is faster than that of the inner layer.

As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

By “inner” is merely meant that the bioerodable polymeric layer is inner relative to the outer bicerodable polymeric layer-not that it is necessarily the innermost layer of the device. Similarly, by “outer” is meant that the bioerodable polymeric layer is outer relative to the inner bioerodable polymeric layer-not that it is necessarily the outermost layer of the device.

Thus, in some embodiments, the outer bioerodable polymeric layer is not the outermost layer of the device, and at least one layer is provided over the outer bioerodable polymeric layer. However, for reasons which will be better understood from the discussion to follow, in these embodiments, no layer should be disposed over the outer layer that has a bioerosion rate that is slower than the bioerosion rate of the outer layer (e.g., a polymeric layer should not be provided over the outer layer which is biostable or which is less bioerodable than the outer layer).

As used herein a “polymeric component” of a given layer is that portion of the layer that is made up of polymers (e.g., a single type of polymer or a combination of two or more types of polymers).

Similarly, a therapeutic agent component of a layer is that portion of the layer that is made up of therapeutic agents. (e.g., a single type of therapeutic agent or a combination of two or more types of therapeutic agents).

As used herein, a polymer is “biodegradable” if it undergoes bond cleavage along the polymer backbone in vivo, regardless of the mechanism of bond cleavage (e.g., enzymatic breakdown, hydrolysis, oxidation, etc.).

Bioerosion is a result of biodegradation (as well as other in vivo disintegration processes such as dissolution, etc.) and is characterized by loss of the original mass of the biodegradable component over time.

Both the inner and outer layers are adapted to be substantially completely bioeroded (i.e., 95 wt % to 97.5 wt % to 99 wt % or more wt % of each region bioerodes in vivo over the period that the device is designed to reside in a patient.

Examples of medical devices benefiting from the present invention include implantable or insertable medical devices from which one or more therapeutic agents may be delivered, for example, catheters (e.g., urological or vascular catheters such as balloon catheters and various central venous catheters), guide wires, balloons, filters (e.g., vena cava filters and mesh filters for distil protection devices), stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, embolization devices including cerebral aneurysm filler coils (including Guglilmi detachable coils and metal coils), embolic agents, hermetic sealants, septal defect closure devices, myocardial plugs, patches, pacemakers, lead coatings including coatings for pacemaker leads, defibrillation leads, and coils, ventricular assist devices including left ventricular assist hearts and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, orthopedic prosthesis such as bone grafts, bone plates, joint prostheses, orthopedic fixation devices such as interference screws in the ankle, knee, and hand areas, tacks for ligament attachment and meniscal repair, rods and pins for fracture fixation, screws and plates for craniomaxillofacial repair, dental implants, as well as various other devices that are implanted or inserted into the body and from which therapeutic agent is released.

The medical devices of the present invention thus include, for example, implantable and insertable medical devices that are used for systemic treatment, as well as those that are used for the localized treatment of any mammalian tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, ears, spine, nervous system, lungs, trachea, esophagus, intestines, stomach, brain, liver and pancreas, skeletal muscle, smooth muscle, breast, dermal tissue, cartilage, tooth and bone. As used herein, “treatment” refers to the prevention of a disease or condition, the reduction or elimination of symptoms associated with a disease or condition, or the substantial or complete elimination of a disease or condition. Preferred subjects are vertebrate subjects, more preferably mammalian subjects and more preferably human subjects.

Specific examples of medical devices include coronary stents that are commonly inserted or implanted into the coronary artery after a procedure such as percutaneous transluminal coronary angioplasty (“PCTA”). Such stents are used to maintain the patency of the coronary artery by supporting the arterial walls and preventing reclosure or collapse thereof, which can occur after PCTA. Metals such as stainless steel or nitinol are commonly used for this purpose as they are strong and have good vascular biocompatibility. These stents can also be adapted to locally release one or more therapeutic agents at the site of implantation. As noted above, such drug eluting coronary stents are commercially available, for example, from Boston Scientific Corp. (TAXUS), Johnson & Johnson (CYPHER). These stents release antiproliferative agents (e.g., paclitaxel, sirodlimus) to inhibit re-narrowing or restenosis of the blood vessel after implantation of the stent. Because materials such as stainless steel are not themselves particularly useful as drug delivery reservoirs, polymer coatings are employed this purpose.

The polymers in these stents are biostable. As previously indicated, however, biodegradable polymers have certain benefits over biostable polymers. For example, the problem of long-term drug entrapment and release is addressed. In this regard, it is currently believed that an antiproliferative drug such as paclitaxel is needed primarily during the initial stages of healing and remodeling, which begin soon after angioplasty, but that the long term presence of such an antiproliferative drug within the vasculature may delay formation of a fully functional endothelium. In addition, a biodegradable polymer coating allows for the ultimate bioerosion of the polymer, which (a) addresses long term effects that may be associated with polymers (e.g., foreign body effects, etc.) and (b) leaves behind a bare metal stent, which is known to be amenable to endothelialization.

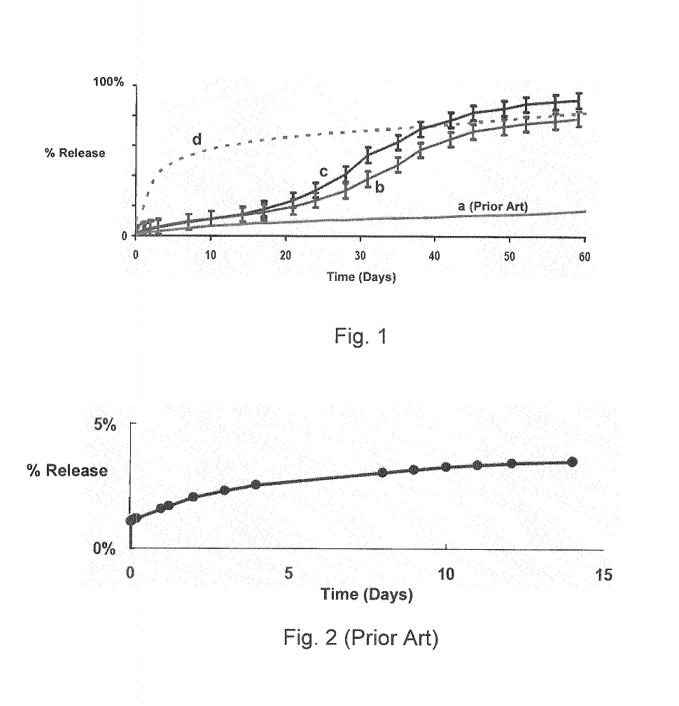

One potential disadvantage to switching from a biostable polymeric coating to a biodegradable polymeric coating is that the drug delivery profile of the coating may be dramatically altered. For example, as seen from FIG. 1 (curve a) and FIG. 2 above, a polymeric coating containing SIBS and paclitaxel provides an initial burst of paclitaxel, followed by a reduction in paclitaxel release after the initial burst. Such release profiles have been shown to be effective in clinical practice. As noted above, it is believed that providing substantial amounts of an antiproliferative drug such as taxol is most desirable during the initial stages of healing and remodeling. However, after this initial period, it is desirable that the drug either cease to be released, or be released in only minute amounts.

Were a layer consisting of a biodegradable polymer and paclitaxel to a have a release profile of this nature, it would look something like that illustrated in curve d of FIG. 1. Curve d of FIG. 1 is similar to curve a of FIG. 1 and FIG. 2 in that it provides an initial burst of paclitaxel, followed by a gradual leveling off. However, Curve d displays a much higher ultimate % release (virtually 100%) due to the use of the biodegradable polymer.

In practice, however, biodegradable polymer typically do not yield release profiles like those shown in curve d of FIG. 1. For example, drug profiles are shown in FIG. 1 for a drug eluting stent, which consists of a 16 mm stainless steel Liberte WH stent (Boston Scientific Corp.) provided with a coating that contains 95 wt % PGLA (50:50 co-polymer ratio with a mixture of both L and D-Lactide) and 5 wt % paclitaxel coated from chloroform. Samples tested include both sterile/expanded samples (represented by curve c) and pre-sterile/non-expanded samples (represented by curve b). Rather than providing an initial burst of paclitaxel, these stents provide a sigmoidal drug release profile in which drug release is tightly controlled in the early stages of polymer degradation, but whose elution kinetics actually increase as the polymer loses molecular weight during the more advanced stages of the degradation process.

By way of background and without wishing to be bound by theory, drugs entrapped within biodegradable polymer matrices are released at rates that are controlled by the diffusion of the drug through the polymer matrix and the degradation of the polymer matrix, among other factors. Polymer biodegradation arises from bond cleavage along the polymer backbone in vivo, which can occur from a variety of bond cleavage mechanisms (e.g., hydrolysis, enzymatic breakdown, etc.), and requires penetration of water (and in some instances other species such as catalysts) into the polymer. Depending on the bioerosion mechanism, polymers can undergo surface erosion, bulk erosion or a combination of both. As a specific example, hydrolysis occurs in biodegradable polymers such as polyanhydrides, polyorthoesters and polyesters, when water contacts the same. If hydrolysis proceeds quickly relative to the rate of water penetration into the polymer bulk, surface erosion will predominate. If hydrolysis proceeds slowly relative to the rate of water penetration into the polymer bulk, bulk erosion will predominate. Thus polymers having relatively high rates of hydrolysis, such as polyanhydrides and polyorthoesters, are commonly referred to as surface eroding polymers, whereas polymers having relatively slow rates of hydrolysis, such as polyesters, are commonly referred to as bulk eroding polymer. Whether erosion is predominantly surface erosion or bulk erosion will also depend upon the physical dimensions of the polymeric region as well. For example, if a polymer layer is made thick enough, the polymer layer will undergo some surface erosion, even if the polymer has relatively low rate of hydrolysis. Conversely, if a polymer layer is made thin enough, the polymer layer will undergo some bulk erosion, even if the polymer has relatively high rate of hydrolysis.

Sigmoidal shaped drug release profiles such as those in curves b and c of FIG. 1 are common for bioerodable drug release layers, particularly those which undergo substantial bulk erosion and which contain sufficiently hydrophobic drugs in sufficiently low amounts, such that drug release depends largely upon the bioerosion of the drug release layer.

The present invention, on the other hand, allows for the creation of medical devices which have release profiles that are more akin to the release profile of FIG. 1d, than to that of FIGS. 2b and 2c.

This is accomplished by providing a medical device that comprises: (a) a substrate, (b) an inner bioerodable polymeric layer over the substrate that comprises (i) 80 wt % or more of an amorphous biodegradable polymeric component and (ii) 20 wt % or less of a therapeutic agent component, and (c) an outer bioerodable polymeric layer over the inner bioerodable polymeric layer that comprises (i) 80 wt % or more of an amorphous biodegradable polymeric component and (ii) 20 wt % or less of a therapeutic agent component. The composition of the amorphous biodegradable polymeric component of the inner layer may be the same as or different from that of the outer layer. The composition of the therapeutic agent component of the inner layer may be the same or different from that of the outer layer.

In the present invention, the compositions of the inner and outer bioerodable polymeric layers differ such that the outer bioerodable polymeric layer has a bioerosion rate that is faster than that of the inner bioerodable polymeric layer. The composition of the inner layer may differ from that of the outer layer, for example, because the composition of the amorphous biodegradable polymeric component of the inner layer is different from that of the outer layer. For instance, as seen from the Examples below, the polymers used in the inner and outer layers may differ in terms of monomer content, monomer ratio, molecular weight, and so forth. The composition of the inner layer may also differ from that of the outer layer, for example, because the ratio of the polymeric component to the therapeutic agent component differs between the layers, among other possibilities.

One possible effect of this combination of layers (among many) is illustrated schematically in FIG. 8. Curve a in this drawing corresponds to the release profile associated with the inner layer, curve b corresponds to the release profile associated with the outer layer, and curve c corresponds to the release profile produced by the inner and outer layers in combination. As can be seen from FIG. 8, the combination of the inner and outer layers provides a drug delivery profile in which drug is released at a relatively high rate early in the delivery profile, in which the drug is released at a relatively lower rate at a later point the in delivery profile, and in which drug release ceases upon complete bioerosion of the polymeric layers. Thus a drug delivery profile is achieved which differs substantially from that of a single layer.

Whether or not a given layer bioerodes at a rate that is greater than another layer can readily be determined by those of ordinary skill in the art, for example, by forming a layer of each composition on a substrate and then implanting or inserting the substrate into a subject. For example, the time required to substantially complete bioerosion of the inner layer may range from 2 to 5 to 10 to 20 times the time required to substantially complete bioerosion of the outer layer.

Whether or not a polymeric component is amorphous can be determined by subjecting the polymeric component to standard x-ray crystallography techniques. In certain embodiments, all polymers within all biocrodable polymeric layers of the device are amorphous polymers. The use of amorphous polymeric components can be advantageous relative to semi-crystalline polymeric components in that semi-crystalline polymers are known to degrade non-homogeneously, with the amorphous regions degrading at a rate faster than the crystalline regions.

In certain embodiments of the invention, the inner and outer layer may independently contain, for example, (a) from 80 wt % to 90 wt % to 95 wt % to 97.5 wt % to 99 wt % or more of at least one biodegradable polymer and (b) from 1 wt % or less to 2.5 wt % to 5 wt % to 10 wt % to 20 wt % of at least one therapeutic agent. The therapeutic agent content of the inner and outer layers is held to 20 wt % or less to avoid the drug release being controlled by the drug content.

As is well known, “polymers” are molecules that contain multiple copies (e.g., 5 to 10 to 25 to 50 to 100 to 250 to 500 to 1000 or more copies) of one or more constitutional units, commonly referred to as monomers. Polymers may take on a number of configurations, which may be selected, for example, from cyclic, linear, branched and networked (e.g., crosslinked) configurations. Branched configurations include star-shaped configurations (e.g., configurations in which three or more chains emanate from a single branch point, such as a seed molecule), comb configurations (e.g., configurations having a main chain and a plurality of side chains), dendritic configurations (e.g., arborescent and hyperbranched polymers), and so forth. As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating), and block copolymers.

Examples of biodegradable polymers for use in the present invention may be selected from suitable members of the following, among many others: (a) polyester homopolymers and copolymers such as polyglycolide (PGA), poly-D-lactide (PLA) including poly-L-lactide, poly-D-lactide, poly-D,L-lactide, poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-glycolide) (PLGA), poly(lactide-co-delta-valerolactone), poly(lactide-co-epsilon-caprolactone), poly(lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], and poly(sebacic acid-co-fumaric acid), among others, (b) poly(ortho esters) such as those synthesized by copolymerization of various diketene acetals and diols, among others, (c) polyanhydrides, such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others; and (d) amino-acid-based polymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and Ea diphenol selected, for instance, from ethyl, butyl, hexyl, octyl and bezyl esters of desamrinotyrosyl-tyrosine), and tyrosine-, leucine- and lysine-based polyester-amides; specific examples of tyrosine-based polymers include includes polymers that are comprised of a combination of desaminotyrosyl tyrosine hexyl ester, desaminotyrosyl tyrosine, and various di-acids, for example, succinic acid and adipic acid, among others.

The present invention thus includes medical devices in which the inner and outer layers are based on biodegradable polyesters, including those from the PGA/PLA/PLGA polymer family, among others. Factors affecting the rate by which polyesters erode, including PLA polymers such as poly(1-lactic acid) (PLLA) and poly(d,l-lactic acid) (PDLLA), PGA polymers, as well as poly(1-lactic acid-co-glycolic acid) (PLLGA) and poly(d,l-lactic acid-co-glycolic acid) (PDLLGA) copolymers, among other family members, include the following: the monomer composition and molecular weight of the polymer, the polymer crystallinity, the concentration and relative hydrophilicity/hydrophobicity of drugs and other optional agents within the layer, and the porosity and dimensions (e.g., thickness) of the layer, among other factors.

In this regard, monomer hydrophilicity can affect erosion rates. For example, PLA erodes slower than PGA. Similarly, for copolymers of lactic acid and glycolic acid (PLGA), higher amounts of lactic acid lead to slower rates of erosion. These effects are thought to be due to the fact that lactic acid is more hydrophobic than glycolic acid.

Higher molecular weight polymers have been shown to erode more slowly than lower molecular weight polymers. For example, in some embodiments, the same polymer is used in the inner and outer layers, but the inner layer has a number average molecular weight that ranges from 1.5 to 2 to 5 to 10 to 20 or more times that of the outer layer.

In addition, crystalline polymers tend to erode more slowly than amorphous polymers. This is believed to be a consequence of the fact that polymers with higher degrees of crystallinity resist water intrusion to a greater degree than polymers with lower degrees of crystallinity. For example, PLLA, which is crystalline, erodes more slowly than PDLLA, which is amorphous. For semi-crystalline polyesters, degradation is thought to first occur in the amorphous domains, followed by the crystalline domains, which in addition to causing non-homogeneous degradation, results in an increase in the overall crystallinity of the polymer as degradation advances.

As noted above, in many embodiments, it is preferred that the biodegradable polymeric layers of the present invention contain an amorphous polymeric component. For example, poly(1-lactic acid) (PLLA) and polyglycolic acid (PGA) are crystalline. Poly(d,l-lactic acid) (PDLLA), on the other hand, is amorphous. The crystallinity of members of the PGA/PLA/PLGA polymer family depends upon the relative amounts of the monomers forming the same. For example, poly(1-lactic acid-co-glycolic acid) (PLLGA) is amorphous over a composition range of from 25 to 70 mol % glycolic acid, whereas poly(d,l-lactic acid-co-glycolic acid) (PDLLGA) is amorphous over a composition range of from 0 to 70 mol % glycolic acid.

More generally, where poly(lactic acid-co-glycolic acid) (PLGA) is employed in the layers of the invention, whether the lactic acid is d-lactic acid, 1-lactic acid or a mixture of d- and 1-lactic acid, the mol % of lactic acid in PLGA for either the inner or the outer layer may range from 0 to 100 mol %, for example, ranging from 30 mol % to 40 mol % to 50 mol % to 75 mol % to 85 mol % to 90 mol % to 95 mol % to 99 mol % to 100 mol %.

Further background information on the above polymers can be found in Xinyin Liu, Drug Delivery Systems Based on Polymer Blends: Synthesis, Characterization & Application, Ph.D. thesis, Drexel University, September 2003, Chapter 2, and references cited therein. See also, e.g., A. Porjazoska et al., “Poly(lactide-co-glycolide) microparticles as systems for controlled release of proteins—Preparation and characterization,” Acta Pharm. 54 (2004) 215-229, and references cited therein. “Therapeutic agents,” “drugs,” “pharmaceutically active agents,” “pharmaceutically active materials,” and other related terms may be used interchangeably herein. These terms include genetic therapeutic agents, non-genetic therapeutic agents and cells.

Exemplary non-genetic therapeutic agents for use in conjunction with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/ antiproliferative/anti-miotic agents such as paclitaxel, 5-fluorouracil, cisplatin, vinblastine, viricristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promoters; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) alpha receptor antagonist (such as doxazosin, Tamsulosin) and beta receptor agonists (such as dobutamine, salmeterol), beta receptor antagonist (such as atenolol, metaprolol, butoxamine), angiotensin-II receptor antagonists (such as losartan, valsartan, irbesartan, candesartan and telmisartan), and antispasmodic drugs (such as oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, and (y) human apolioproteins (e.g., AI, AII, AIII, AIV, AV, etc.).

Specific examples of non-genetic therapeutic agents include paclitaxel, (including particulate forms thereof, for instance, protein-bound paclitaxel particles such as albumin-bound paclitaxel nanoparticles, e.g., ABRAXANE), sirolimus, everolimus, tacrolimus, Epo D, dexamethasone, estradiol, halofuginone, cilostazole, geldanamycin, ABT-578 (Abbott Laboratories), trapidil, liprostin, Actinomcin D, Resten-NG, Ap-17, aboiximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Exemplary genetic therapeutic agents for use in conjunction with the present invention include anti-sense DNA and RNA as well as DNA coding for the various proteins (as well as the proteins themselves): (a) anti-sense RNA, (b) tRNA or rRNA to replace defective or deficient endogenous molecules, (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor a, hepatocyte growth factor and insulin-like growth factor, (d) cell cycle inhibitors including CD inhibitors, and (e) thymidine kinase (“TKE”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors, for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or microparticles, with and without targeting sequences such as the protein transduction domain (PTD).

Cells for use in conjunction with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, ( ) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and TCAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites, such as purine analogs (e.g., 6-mercaptopurine or cladribine, which is a chlorinated purine nucleoside analog), pyrimidine analogs (e.g., cytarabine and 5-fluorouracil) and methotrexate , nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin (sirolimus) and its analogs (e.g., everolimus, tacrolimus, zotarolimus, etc.), cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Further additional therapeutic agents useful for the practice of the present invention are also disclosed in U.S. Pat. No. 5,733,925 assigned to NeoRx Corporation, the entire disclosure of which is incorporated by reference.

A variety of materials may be used as substrate materials for the medical devices of the present invention. Examples of such materials include non-metallic materials such as ceramics, homopolymers, copolymers, and polymer blends. Examples of such materials also include metallic materials, such as metals (e.g. Ti, Ta), metal alloys comprising iron and chromium (e.g., stainless steels, including platinum-enriched radiopaque stainless steel), alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N) and alloys comprising cobalt, chromium, tungsten and nickel (e.g., L605), alloys comprising nickel and chromium (e.g., inconel alloys). Composites of two or more of the forgoing (e.g., polymer-ceramic composites, polymer-metal composites, metal-ceramic composites, etc.) may also be employed. Materials having both super elastic and shape-memory characteristics, for example, alloys comprising nickel and titanium (e.g., Nitinol) may be beneficial in certain embodiments.

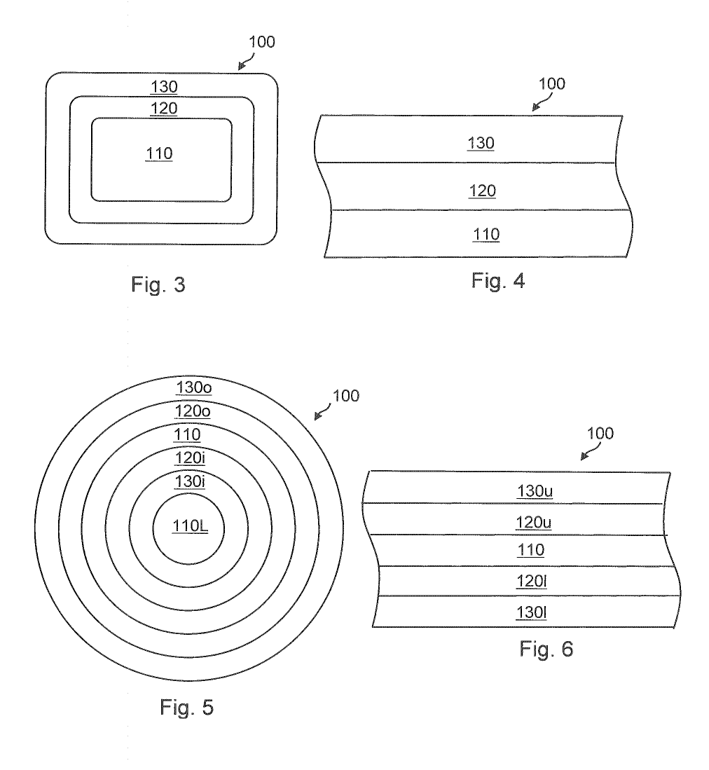

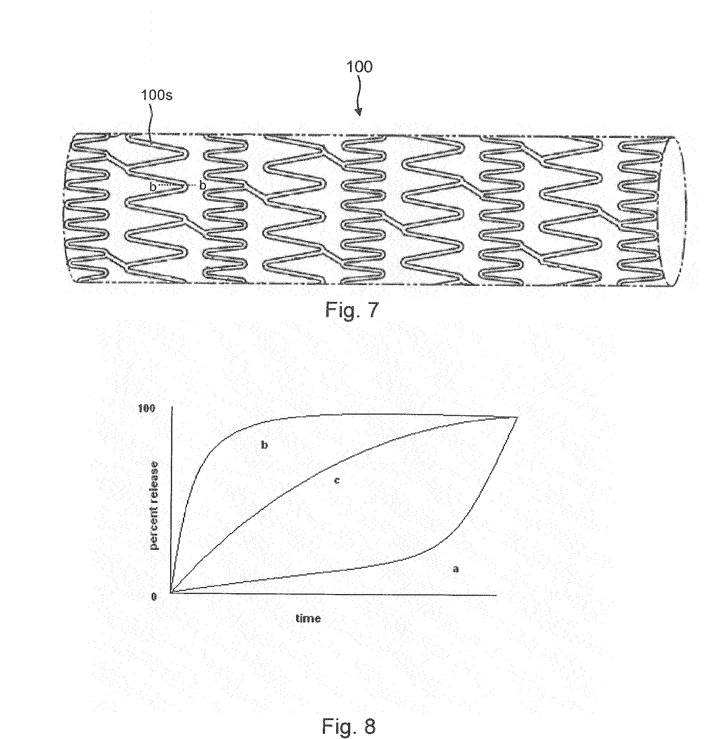

Examples of a few of the many possible configurations for the medical devices of the invention will now be described in conjunction with the FIGS. 3-7. FIG. 3, is a schematic view of a medical device or portion thereof 100 that is substantially rectangular in cross-section. FIG. 3 may correspond, for example, to a cross-section of a stent strut, such as that taken along line b--b of strut 110s of stent 100 of FIG. 7, among many other possibilities. The device or portion thereof 100 comprises a substrate 110, an inner layer 120 provided over the substrate 110, and an outer layer 130 provided over the inner region 120.

Another specific embodiment will now be described with reference to FIG. 4, which is a partial schematic cross-sectional view of a substantially planar medical device or portion thereof 100. The device or portion thereof 100 comprises a substantially planar substrate 110, an inner layer 120 provided over the substrate 110, and an outer layer 130 provided over the inner region 120.

Another embodiment of the invention is described with reference to FIG. 5, which is a schematic view of a medical device or portion thereof 100 that is substantially annular in cross-section. The device or portion thereof 100 has an inner lumen 100L and comprises an annular substrate 110, an inner layer 120i disposed on an inner surface of the substrate 110, an inner layer 120o disposed on an outer surface of the substrate 110, an outer layer 130i disposed on an inner surface of the inner layer 120i, and an outer layer 130o disposed on an outer surface of the inner layer 120o. In related embodiments, the outer layer 130o and inner layer 120o may be eliminated (e.g., to direct drug delivery to the luminal surface of the device). In further related embodiments, the outer layer 130i and inner layer 120i may be eliminated (e.g., to direct drug delivery to the abluminal surface of the device).

Still another specific embodiment will now be described with reference to FIG. 6, which illustrates a partial cross section of a generally planar medical device or portion thereof 100. The device or portion thereof 100 comprises a substantially planar substrate 110, an inner layer 120u disposed on an upper surface of the substrate 110, an inner layer 1201 disposed on a lower surface of the substrate 110, an outer layer 130u disposed on an upper surface of the inner layer 120u, and an outer layer 1301 disposed on a lower surface of the inner layer 1201.

Numerous techniques are available for forming the medical devices (or portions thereof) of the invention.

For example, in some embodiments, solvent-based techniques are used to form one or more of the various regions of the devices of the present invention (e.g., the substrate, the inner layer, the outer layer, any additional layers). Using these techniques, regions can be formed by first providing a solution that contains the chemical species that make up the regions (e.g., polymer, therapeutic agent, and/or other chemical species), dissolved or dispersed therein, and subsequently removing the solvent system. The solvent system that is ultimately selected will contain one or more solvent species, which may be selected based on their ability to dissolve or disperse the various chemical species, as well as other factors, including drying rate, surface tension, etc. Examples of solvent-based techniques include solvent casting techniques, spin coating techniques, web coating techniques, solvent spraying techniques, dipping techniques, techniques involving coating via mechanical suspension including air suspension, ink jet techniques, electrostatic techniques, and combinations of these processes, among others.

In other embodiments, thermoplastic processing techniques are used to form one or more of the various regions of the present invention. Using these techniques, regions can be formed by first providing a melt that contains the chemical species that make up the regions, and subsequently cooling the melt. Examples of thermoplastic techniques include compression molding, injection molding, blow molding, spinning, vacuum forming and calendaring, as well as extrusion into sheets, fibers, rods, tubes and other cross-sectional profiles of various lengths. Using these and other thermoplastic processing techniques, a variety of regions can be formed.

In some embodiments of the invention, a solution (where solvent-based processing is employed) or melt (where thermoplastic processing is employed) is applied to an underlying region. For example, the underlying region may correspond to all or a portion of an implantable or insertable medical device substrate. For instance, inner and outer layers may be applied sequentially to the substrate, inner and outer layers may be coextruded together onto a substrate, and so forth. The underlying region can also be, for example, a template, such as a mold, from which subsequently applied region(s) may be removed after solidification. In still other examples, various regions may be formed without the aid of a substrate. For example, the substrate, inner layer and outer layer may be coextruded together.

Other ways of forming medical devices in accordance with the present invention will become readily apparent to those of ordinary skill upon review of the above description of the invention.

### EXAMPLES

In the embodiments of Examples 1 and 2 below, PLGA drug eluting coating systems for stents are described, each of which consists of two layers of PLGA with higher drug loadings in the outer layer than in the inner layer. The higher drug loading leads to higher bioerosion rates.

### Example 1

A 16 mm Liberte WH stent (Boston Scientific, Natick, Mass., USA) is provided with two layers having a total coating weight of 400 μg. The inner layer is a 200 μg layer that consists of 1 wt% paclitaxel (2 kg) and the remainder PLGA (50:50). The outer layer is a 200 μg layer that consists of 9 wt % paclitaxel (18 μg) and the remainder PLGA (50:50). Total paclitaxel loading is 20 μg.

### Example 2

A 16 mm Liberte WH stent is provided with two layers having a total coating weight of 400 μg. The inner layer is a 300 μg layer that consists of 1.33 wt % paclitaxel (4 μg) and the remainder PLGA (50:50). The outer layer is a 100 μg layer that consists of 16 wt % paclitaxel (16 μg) and the remainder PLGA (50:50). Total paclitaxel loading is 20 μg.

In the embodiments of Examples 3 and 4 below, PLGA drug eluting coating systems for stents are described, each of which consists of two layers of PLGA with a faster bioeroding polymer carrier in the outer layer than in the inner layer. As noted above, faster bioerosion may be achieved, for example, by varying the monomer fraction within the copolymer, by varying the molecular weight of the copolymer, and so forth.

### Example 3

A 16 mm Liberte WH stent is provided with two layers having a total coating weight of 400 μg. The inner layer is a 200 μg layer that consists of 5 wt % paclitaxel (10 g) and the remainder PLGA (85:15). The outer layer is a 200 μg layer that consists of 5 wt % paclitaxel (10 μg) and the remainder PLGA (50:50). Total paclitaxel loading is 20 μg.

### Example 4

A 16 mm Liberte WH stent is provided with two layers having a total coating weight of 400 μg. The inner layer is a 200 μg layer that consists of 5 wt % paclitaxel (10 μg) and the remainder PLGA (85:15, high molecular weight, e.g., 85,000 Daltons). The outer layer is a 200 μg layer that consists of 5 wt % paclitaxel (10 μg) and the remainder PLGA (85:15, low molecular weight, e.g., 5,000 Daltons). Total paclitaxel loading is 20 μg.

In the embodiment of Example 5 below, a PLGA drug eluting coating system for a stent is described which consists of two layers of PLGA, with a bioeroding polymer carrier and a greater amount of therapeutic agent in the outer layer, thereby providing a further level of control of elution profile.

### Example 5

A 16 mm Liberte WH stent is provided with two layers having a total coating weight of 400 μg. The inner layer is a 100 μg layer that consists of 2 wt % paclitaxel (4 μg) and the remainder PLGA (85:15). The outer layer is a 300 μg layer that consists of 5.33 wt % paclitaxel (16 μg) and the remainder PLGA (50:50). Total paclitaxel loading is 20 μg.

Although various embodiments are specifically illustrated and described herein, it will be appreciated that modifications and variations of the present invention are covered by the above teachings and are within the purview of the appended claims without departing from the spirit and intended scope of the invention.

1. A medical device comprising (a) a substrate, (b) an inner bioerodable polymeric layer over the substrate that comprises (i) 80 wt % or more of a first amorphous biodegradable polymeric component and (ii) 20 wt % or less of a therapeutic agent component, and (c) an outer bioerodable polymeric layer over the inner biocrodable polymeric layer that comprises (i) 80 wt % or more of a second amorphous biodegradable polymeric component and (ii) 20 wt % or less of the therapeutic agent component, wherein the first and second amorphous biodegradable polymeric components are the same or different, and wherein the inner and outer bioerodable polymeric layers differ in composition such that the outer bioerodable polymeric layer has a bioerosion rate that is faster than that of the inner bioerodable polymeric layer.

2. The medical device of claim 1, wherein the first and second amorphous biodegradable polymeric components are different.

3. The medical device of claim 2, wherein the first amorphous biodegradable polymeric component comprises a first polymer having a first monomer content and wherein the second amorphous biodegradable polymeric component comprises a second polymer having a second monomer content that differs from the first monomer content.

4. The medical device of claim 3, wherein the first polymer comprises a monomer species that is not found in the second polymer or wherein the second polymer comprises a monomer species that is not found in the first polymer.

5. The medical device of claim 4, wherein the first polymer is poly(1-lactic acid-co-glycolic acid) or poly(d,l-lactic acid-co-glycolic acid) and wherein the second polymer is poly(d,l-lactic acid).

6. The medical device of claim 3, wherein the first and second polymers are copolymers comprising first and second monomers and wherein the ratio of the first monomer to the second monomer differs between the first and second polymers.

7. The medical device of claim 6, wherein the first and second polymers are poly(1-lactic acid-co-glycolic acid) or poly(d,l-lactic acid-co-glycolic acid), and wherein the mol % of lactic acid within the first polymer is less than that within the second polymer.

8. The medical device of claim 7, wherein the amount of d,l-lactic acid within the first polymer ranges from 30 to 85 mol % and wherein the amount of d,l-lactic acid within the second polymer ranges from 30 to 100 mol %.

9. The medical device of claim 1, wherein the first and second amorphous biodegradable polymeric components are the same and wherein the wt % of the therapeutic agent component within the outer bioerodable polymeric layer is greater than that of the inner bioerodable polymeric layer.

10. The medical device of claim 1, wherein the first and second amorphous biodegradable polymeric components are different and wherein the wt % of the therapeutic agent within the outer bioerodable polymeric layer is greater than that of the inner bioerodalble polymeric layer.

11. The medical device of claim 1, wherein the inner and outer layers each comprises 10 wt % or less of the therapeutic agent component.

12. The medical device of claim 1, wherein the wt % of the therapeutic agent component within the outer bioerodable polymeric layer is at least 2 times that of the inner bioerodable polymeric layer.

13. The medical device of claim 1, wherein the outer biocrodable polymeric layer is the outermost layer in the medical device.

14. The medical device of claim 1, wherein no region within the medical device comprises a crystalline or semi-crystalline biodegradable polymeric component.

15. The medical device of claim 1, wherein the substrate is metallic.

16. The medical device of claim 1, wherein the substrate is a vascular stent.

17. The medical device of claim 1, wherein the device comprises no polymeric layers other than the inner and outer bioerodable polymeric layers.

18. The medical device of claim 1, wherein the inner and outer bioerodable polymeric layers are non-porous.

19. The medical device of claim 1, wherein the first and second amorphous biodegradable polymeric components consist of biodegradable polyesters.

20. The medical device of claim 19, wherein the biodegradable polyesters comprise lactic acid monomers or a combination of lactic acid and glycolic acid monomers.

21. The medical device of claim 1, wherein the therapeutic agent component comprises a plurality of differing therapeutic agents.

22. The medical device of claim 1, wherein the therapeutic agent component is selected from anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, agents that interfere with endogenous vasoactive mechanisms, and combinations thereof

23. The medical device of claim 1, wherein the medical device is a vascular medical device.

24. The medical device of claim 2, wherein the first amorphous biodegradable polymeric component comprises a first polymer having a first monomer content, wherein the second amorphous biodegradable polymeric component comprises a second polymer having a second monomer content that is the same as the first monomer content, and wherein the second polymer has a number average molecular weight that is at least 10 kDa greater than the first polymer.
